(19) United States
(12) Patent Application Publication
(10) Pub. No.: US 2009/0012409 A1
(43) Pub. Date: Jan. 8, 2009

(54) Determining Blood Pressure

(76) Inventors: Gerrit Roenneberg, Darmstadt (DE); Fred Schnak, Kronberg (DE); Dieter Wunder, Schotten (DE)

Correspondence Address: FISH & RICHARDSON PC, P.O. BOX 1022, MINNEAPOLIS, MN, 55440-1022, US

(21) Appl. No.: 11/631,717

(22) PCT Filed: Jun. 29, 2005
(86) PCT No.: PCT/EP05/06980

§ 371 Date: Aug. 26, 2008

(30) Foreign Application Priority Data
Jul. 5, 2004 (DE) 10 2004 032 579.0

(51) Int. Cl. A61B 5/022

(52) U.S. Cl. 600/485

(57) ABSTRACT

A method and a measuring device for determining blood pressure, pressure signals being detected using a pressure sensor which may be applied to a body part, such as a wrist. The blood pressure is determined by an analysis unit, analyzing the pressure signals and considering signals from an orientation detection unit detecting the position and/or movement and/or acceleration of the body part.

The present invention relates to a method for determining blood pressure and a blood-pressure measuring device

Blood-pressure measurements on the wrist or on the finger often suffer from a lack of measurement precision and insufficient reproducibility. This is caused by the high sensitivity of the measurement in regard to variations of the measurement position, i.e., the individual position of the wrist or the finger in relation to the position of the heart. To obtain exact results, a measurement at heart height is necessary in known measuring devices which are applied to the wrist. However, this is typically only approximately maintained and is perceived as restrictive and impractical by the people on whom the blood-pressure measurement is performed. Imprecision is therefore always inherent in the measurements. In the case of a measurement position deviating from the heart height, the hydrostatic pressure differential corrupts the measurement result by approximately 0.75 mm hg/cm, from which a systematic measurement error may result. In addition, a brief, rather random position variation due to shaking or in our movement, for example, may also result in a second dynamic error, known as movement artifacts, which make the algorithmic analysis of the actual measured variable significantly more difficult, if not even impossible.

U.S. Pat. No. 5,770,879 therefore suggests a blood-pressure measuring device in which the inclination of the blood-pressure measuring device attached to the wrist is determined using an angle sensor, which is taken as a measure of the height of the blood-pressure measuring device in relation to the heart. If the angle or the height is too high or too low, corresponding error messages are output. The operator of the blood-pressure measuring device is to first begin the actual measuring procedure when an error message is no longer output and/or the correct position of the blood-pressure measuring device in relation to the heart is indicated. Furthermore, JP-A-7-143970 discloses a blood-pressure measuring device in which the height of the measuring device attached to the wrist in relation to the heart is also determined and displayed via an inclination angle sensor. The height detection may be tailored individually to the user, in that the inclination angle sensor is adjustable in regard to its zero position, which corresponds to the correct positioning of the blood-pressure measuring device. However, there is no monitoring of the position, movement, and/or acceleration of the body part during the actual measurement in these achievements of the object. Rather, it is presumed that the attitude assumed with the aid of the position sensor before the actual measurement is also maintained during the measurement. However, experience shows that this is not the case.

JP-A-5-200004 also discloses a blood-pressure measuring device, in which movements of the arm to which the blood-pressure measuring device is attached are detected with the aid of an acceleration sensor. If the detected movement or acceleration exceeds a predefined value, the actual measuring procedure is delayed until the movement or acceleration falls below the predefined value. If necessary, the measurement is performed again. Another blood-pressure measuring device is disclosed in WO 98/08429, in which the orientation, movement, and acceleration of the body part on which the blood pressure is measured are detected parallel in time to the pressure measurement. The detected blood pressure is then to be corrected in accordance with the detected movement. If too large movements are detected, which prevent precise determination of the blood pressure, the measurement is entirely suppressed.

The measurement precision and reproducibility may be improved using these known blood-pressure measuring devices. However, the practical handling is strongly impaired, since the measurement sequences must often be run through multiple times until a measurement pass is performed completely without errors, i.e., without movements in the correct position of the blood-pressure measuring device.

Furthermore, a blood-pressure measuring device is known from EP 1 041 925 B1, in which the measurement may be suppressed and/or the low informational content of the measurement may be noted in the event of faulty measurement position before, during, or after the measurement.

The present invention is based on the object of specifying an improved method and an improved measuring device for determining the blood pressure, which avoid the disadvantages of the prior art and refine the prior art advantageously. Efficient ability to perform the measurement procedure is preferably to be achieved with high measurement precision and reproducibility.

This object is achieved according to the present invention by a method according to Claim 1. In regard to the device, the stated object is achieved by a measuring device according to Claim 13. Preferred embodiments of the present invention are the subject matter of the subclaims.

It is thus provided according to the present invention that for at least four pressure signals, the particular assigned signal for the position and/or movement and/or acceleration of the body part is detected. The pressure signals are then analyzed individually as a function of the associated value of the position, movement, and/or acceleration of the body part to determine the blood pressure. In regard to the device, for this purpose, the orientation detection unit provides an orientation signal, continuously or synchronized with the signals of the pressure sensor, which indicates the position, movement, and/or acceleration of the body part in the times of the pressure measurements, the analysis unit links the pressure signals of the pressure sensor individually to the associated orientation signals of the orientation detection unit, and only uses the pressure signals to determine the blood pressure as a function of the associated orientation signal. The present invention is therefore based on the idea that the orientation of the body part, possibly including its movement and acceleration, is detected at least four times during the measurement or during the entire blood-pressure measurement, i.e., during the detection of all pressure signals, the entire blood-pressure measurement not being discarded immediately if data was only recorded for a certain part, while the body part was not held in the correct position and/or not sufficiently still. It is decided individually for each pressure signal whether and how it will be used for determining the blood pressure. The blood pressure may thus advantageously be determined extremely precisely if only a few of the measurement values have been recorded in unfavorable conditions, but the sufficiently large remainder has been recorded at setpoint conditions. Multiple passes through the entire measurement routine are avoided (termination of the measurement and restart) if only short, slight shaking has occurred, for example, since the remaining measurement values are sufficiently precise. Alternatively or additionally, it is at least indicated after the measurement if the measurement did not occur at heart height, so that the user receives a notification of the low reproducibility of the measurement result.

In particular, the analysis unit may be implemented in such a way that the pressure signals in which the position and/or movement and/or acceleration of the body part do not lie in the particular predefined setpoint ranges are passed over when determining the blood pressure, so that the blood pressure is determined solely on the basis of the remaining detected pressure signals, in which the position, movement, and/or acceleration of the body part on which the measurement is performed were in the particular predefined setpoint ranges. The singular suppression of pressure measurement values which were recorded in unfavorable conditions significantly increases the precision and reproducibility of the measurement. Simultaneously, the time loss which would be caused by a complete pass through the entire measurement routine once again is avoided. A memory is assigned to the analysis unit, in which the pressure signals and the associated orientation signals, which indicate the position, movement, and/or acceleration of the body part, are initially stored, so that the analysis unit may read out the values accordingly when determining the blood pressure. The pressure signals recorded outside the position, movement, and/or acceleration tolerance ranges may not even be written in the memory. The immediate analysis of the pressure signals in regard to their recording conditions reduces the quantity of data to be processed later when determining the blood pressure from the pressure signals.

Depending on the method of blood pressure determination, the pressure signals detected on the body part are processed further in various ways. One method is oscillometry, in which the strength of the arterial pulsations is determined from the pressure signals. It is calculated for each of these pulsations which pressure was applied to the body part as the strength of the arterial pulsation was registered at the wrist. For this purpose, in a refinement of the present invention, it may be ascertained with the aid of the orientation detection unit whether the measuring device was held at heart height at the time of the occurrence of the particular pulsation and/or was held at heart height during a time interval which is to include the time of the occurrence of the pulsation and/or has moved at the time of the occurrence of the pulsation, so that it was possibly only coincidentally at heart height at the corresponding time and/or it had moved too strongly during a time interval which is to include the time of the occurrence of the pulsation, so that it was not located at heart height at a specific probability or the pressure signal was disturbed by movement artifacts. Furthermore, it may be determined for the cited times whether accelerations of the blood-pressure measuring device exist and/or exceed a specific variable, which may have been generated not only by relative movements of the arm to the body, but rather also by overall movements of the entire body and may negatively influence the measurement result in the same way.

In a refinement of the present invention, the blood-pressure measuring device comprises a display unit for displaying the particular detected position, movement, and/or acceleration of the body part and/or a variable derived therefrom. The display preferably occurs essentially synchronized and/or with only a slight delay to the detection of the position, movement, and/or acceleration of the body part, advantageously also during the measurement procedure. In particular, the display unit gives a feedback signal which indicates to the user of the measuring device whether the is holding the body part correctly or preferably how the is to hold the body part better. In particular in connection with the only singular suppression of measurement values which were recorded during incorrect and/or too restless position of the body part, this simultaneous and/or immediate display has great advantages, since the measurement series may still be saved by correspondingly rapid reaction of the user of the measuring device. Alternatively or additionally, a corresponding feedback signal is displayed after the measurement.

In a refinement of the present invention, the orientation measurement values recorded during the entire measuring cycle are used not only for analyzing the pressure signals, but rather also for controlling the measurement sequence for detecting the cited pressure signals. The body part is regularly subjected to a pressure which changes during the detection of the pressure signals using a pressure sleeve. In a refinement of the present invention, the blood-pressure measuring device comprises a measurement sequence controller which controls the pressure change cycle to which the body part is subjected as a function of the particular detected position, movement, and/or acceleration of the body part. In particular, a pressure change provided in the predefined pressure change cycle may be interrupted and/or delayed if the position, movement, and/or acceleration of the body part lies outside predefined limiting values. As soon as the position, movement, and/or acceleration of the body part detected by the orientation detection unit lies within the predefined limiting values again, the interrupted pressure change cycle is resumed again and/or continued. The pressure change may advantageously be started again using the pressure value which was predefined before the position, movement, and/or acceleration of the body part left the setpoint range. It is thus ensured that each section of the pressure change cycle is passed through completely under setpoint conditions, i.e., the pressure changes are recorded at each time of the pressure change cycle at correct position and/or only sufficiently small movements or accelerations.

The measurement sequence controller advantageously also comprises time detection, in particular the time duration within which the detected position, movement, and/or acceleration values of the body part lie outside the particular predefined setpoint ranges. If this time duration exceeds a predefined limiting value, the measurement sequence controller provides a termination of the measurement cycle. It is thus ensured that no excessively long interruptions of a measurement cycle may occur, which may cause imprecision of the measurement result.

Further advantages, possible applications, and advantageous features of the present invention result from the following description of an exemplary embodiment of the present invention, which is illustrated in the figures of the drawing. All features described or illustrated form the object of the present invention alone or in any arbitrary combination, independently of their summary in the patent claims or their reference back and independently of their formulation and/or illustration in the description and/or in the drawing. In the drawing:

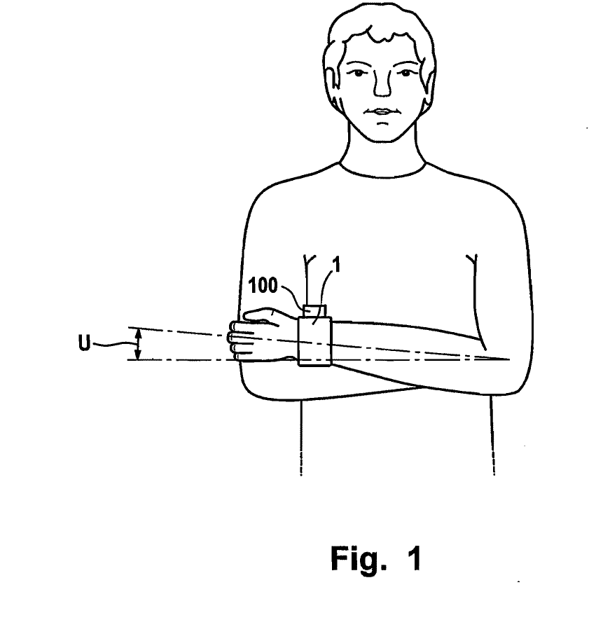

FIG. 1 shows a blood-pressure measuring device corresponding to a preferred embodiment of the present invention, which is applied to a wrist of a person, in a schematic illustration,

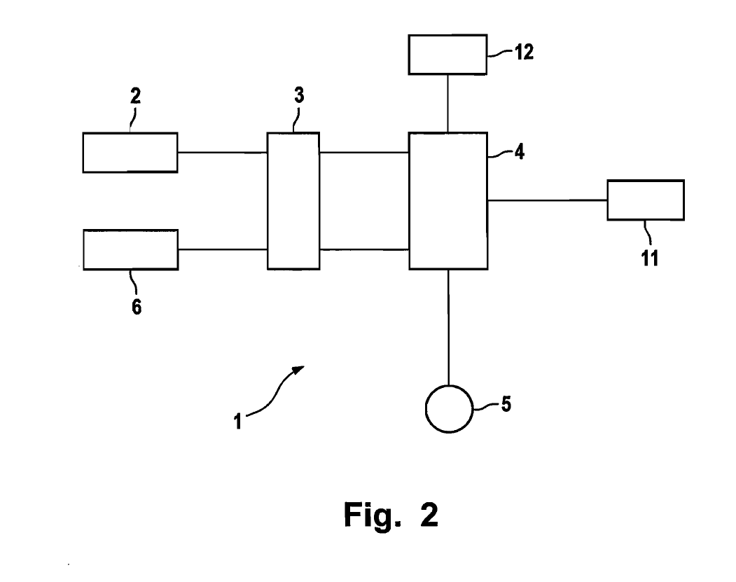

FIG. 2 shows the construction of the blood-pressure measuring device from FIG. 1 in a schematic illustration,

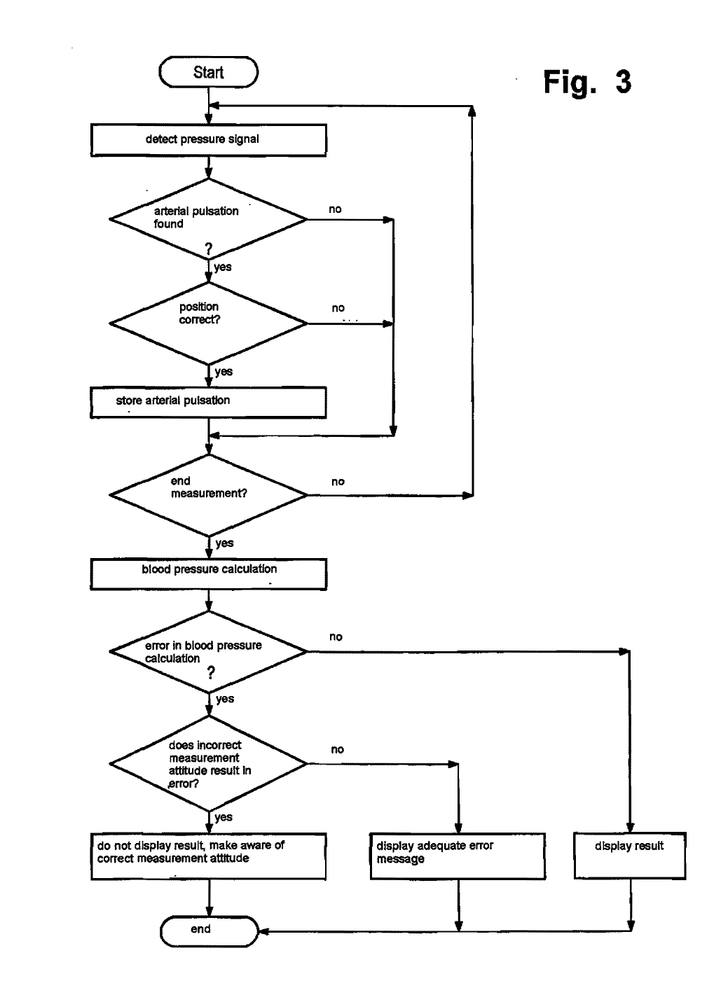

FIG. 3 shows a flowchart of a measurement sequence having checking of the measurement position and movement artifacts if arterial pulsations occur,

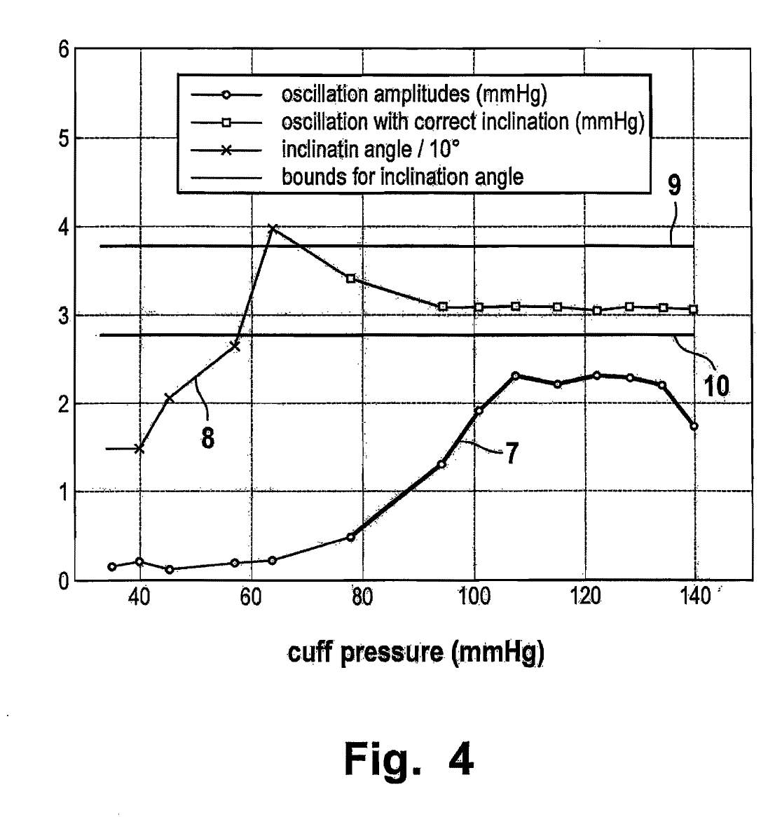

FIG. 4 shows a graph of the arterial pulsations over the sleeve pressure, the inclination angle of the forearm detected in synchronization being plotted in the diagram, and

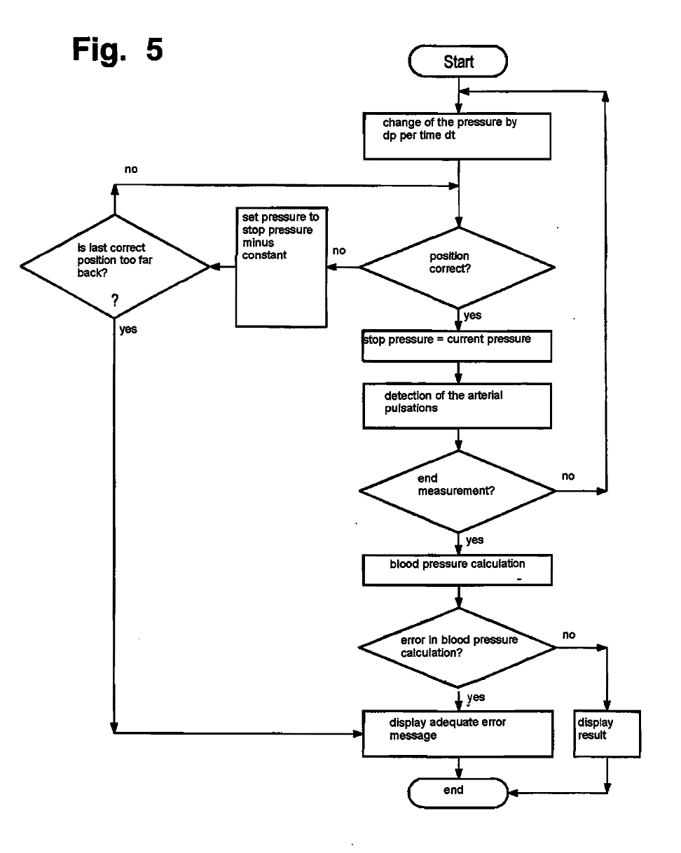

FIG. 5 shows a flowchart of a measurement sequence having direct intervention in the measurement sequence if the optimum measurement position is left and/or if undesired movement artifacts occur.

The blood-pressure measuring device shown in FIG. 1 is implemented to measure the blood pressure on the wrist. As an ideal measurement position, the wrist is directly along the upper body (moved in front of the chest), to find a measurement position at heart height under the direction of the blood-pressure measuring device. It has a sleeve 1 as an application unit, using which a pressure may be applied to a body part, in particular to the interior of the left wrist and/or to the forearm, for signal recording. The sleeve 1 has a bladder for this purpose, which preferably may be inflated using air in order to determine the diastolic, the systolic, and possibly the mean blood pressure and the pulse as the air is released using the oscillometric measuring method. The application unit is in fluid communication with a pressure sensor 2, which is situated in a blood-pressure measuring device housing 100, which is attached to the application unit 1. The pressure sensor 2 (FIG. 2) may be implemented as a capacitive or piezoresistive sensor, for example. The blood-pressure measuring device housing has all other typical components of a blood-pressure measuring device, which are partially listed below.

The pressure sensor 2 is connected via an amplifier and analog/digital converter 3 to a central control and analysis unit 4, which is implemented as a microcontroller or as a digital signal processor, for example, and is provided for the algorithmic analysis of the electrical signal of the pressure sensor 2 and for activating a pressure control unit 5, which is connected to the sleeve 1.

Furthermore, the blood-pressure measuring device has an orientation sensor 6, which may also be connected to the control and analysis unit 4 via a signal preparation unit, which is implemented as an amplifier, and the analog/digital converter 3. The orientation sensor 6 provides a signal which determines the position of the blood-pressure measuring device in relation to the heart, its movement, and its acceleration, and/or makes these values derivable. The orientation sensor 6 may be implemented as an inclination sensor, which detects the inclination of the sleeve 1 and thus the wrist and/or the forearm in relation to the horizontal, i.e., provides an electrical signal which corresponds to the inclination angle u of the wrist to the horizontal (cf. FIG. 1). It may preferably have a movably mounted part such as a pendulum and be provided with a unit, using which the inclination angle u, the velocity, and the acceleration of the movable part are electrically detectable. The velocity and the acceleration may be detected, for example, by electronically generating the first and second derivative of the inclination signal.

If the blood-pressure measuring device is attached to the left wrist as shown in FIG. 1, the control and analysis unit 4 initiates a measurement sequence. For this purpose, the user is guided into a position at heart height using arrow symbols or other display means on the display unit of the blood-pressure measuring device. The control and analysis unit 4 then activates the pressure control unit 5 accordingly, so that the sleeve 1 is first inflated to apply pressure to the wrist. The air in the bladder of the sleeve 1 is then released again to reduce the pressure. A predefined pressure change cycle is passed through in this way. Pressure signals, which correspond to the arterial pulsation, are detected at predefined times using the pressure sensor 2. If the arterial pulsation is not found, the pressure cycle is begun from the beginning if necessary (cf. FIG. 3). Alternatively, the blood pressure is measured simultaneously as the pressure is applied, i.e., during the inflation procedure of the sleeve bladder. The pressure is then released from the sleeve rapidly without further measurement.

FIG. 4 shows the detected pressure signals, which are plotted over the pressure in the sleeve 1, using the points of the graph 7.

During the entire detection of the pressure signals, it is detected using the orientation sensor 6 whether the blood-pressure measuring device and/or the wrist are held in the provided position, approximately at the height of the heart. The orientation signals of the orientation sensor 6, which may correspond to the inclination angle u, are detected in synchronization with the corresponding pressure signals, so that it may be determined for each pressure signal whether the prescribed position has been maintained. In FIG. 4, the second graph 8 shows the detected inclination angle signals at approximately the times of the corresponding pressure signals. In addition, an upper limiting value 9 and a lower limiting value 10 are plotted for the inclination angle. The first five measuring points of the inclination angle are outside the provided setpoint range, which lies between the two limiting values 9 and 10. The corresponding pressure signals of the graph 7 are thus detected at times in which the wrist was not held in the correct position. They are not used for the analysis and determination of the blood pressure. As FIG. 4 shows, only the part of the graph 7 indicated by the thick line is used for determining the blood pressure, since the wrist and the blood-pressure measuring device were held correctly only for the pressure signals which support the curve of this graph section. Only one of the criteria of position, movement, or acceleration is preferably detected.

Although this is not shown in the illustration of FIG. 4, it is obvious that not only the inclination angle per se may be detected and compared to limiting values. Preferably, a movement and/or acceleration of the blood-pressure measuring device is also or alternatively determined during the detection of the pressure signals and compared to corresponding limiting values. The control and analysis unit 4 preferably determines the blood pressure only from the pressure signals in which the inclination angle, its derivative, and its second derivative lie within predefined setpoint ranges.

The user is made aware of the correct measurement attitude if necessary as a function of the signals detected by the orientation sensor 6 by the control and analysis unit 4 via the display unit 11 connected thereto. This may be performed by a request to hold the wrist higher or to hold the wrist still, for example. The number of the pressure signals which are to be ignored for the later determination of the blood pressure may be reduced by a corresponding display on the display unit 11.

As FIG. 5 shows, the control and analysis unit 4 may influence the measurement sequence, in particular the pressure change cycle and the pressure signal detection, even during the measurement itself in the event of the detection of an incorrect measurement attitude and/or undesired movement artifacts. There are various possibilities in principle for this purpose. Firstly, in the event of a continuous change of the pressure which acts on the wrist, this continuous change may be interrupted. For this purpose, the aeration and/or the ventilation procedure may be stopped, which may be caused by closing a valve or stopping the pump. This gives the user time to correct the measurement attitude, which may advantageously be steered appropriately by the display unit 11. If the measurement attitude is correct again and if the correct measurement attitude was also maintained for a specific time if necessary, the continuous pressure change and thus the normal measurement procedure are continued with the detection of the pressure signals.

If a non-continuous, i.e., stepped change of the pressure acting on the wrist is provided, the control and analysis unit 4 may first start the next pressure stage when the measurement attitude is correct and no undesired movement artifacts may be established.

The normal pressure change cycle may be interrupted in both cases in such a way that the pressure applied to the wrist is returned to a value which existed at a moment which occurred at a specific time before leaving the correct measurement position and/or before occurrence of the movement artifacts. In case of an implementation using an air volume whose pressure may be controlled using pump and/or valve, the valve may be opened and/or the pump may be stopped for this purpose for a specific time. In this way, upon reaching the pressure again at which leaving the provided measurement position and/or the undesired movement artifacts previously occurred, the interference of the pressure curve by the measures introduced may be minimized.

If the measurement position is not assumed again within a specific time or if the movement artifacts do not disappear within a specific time, the entire blood-pressure measurement may be terminated.

It is preferably not only indicated by the display unit 11 that the provided measurement position was left and/or undesired movement artifacts have occurred, but rather also that the regular pressure change cycle has not yet been terminated and the user has returned thereto. In this way, the user knows that the measurement continues. The way in which the user is made aware of leaving the regular pressure change and the necessity of some measures for the purpose of again reaching the provided measurement position and/or terminating movement artifacts may vary. This may be performed acoustically via a buzzer or similar means, or tactilely, for example, by a vibration using a pump or valve, and finally visually via a display, colored LEDs, arrow symbols, etc., for example.

The user obtains an indication as to whether the blood-pressure measurement is valid, i.e., usable, or invalid and thus unusable via the validation display during the blood-pressure measurement and/or afterward or after a termination of the blood-pressure measurement because of faulty position (too great a deviation from the heart height), movement (strong movement away from the heart height), and/or acceleration (e.g., shaking during the measurement) of the wrist having the blood-pressure measuring device. This may be shown by a torso displayed on the display on which a forearm is shown at too high a position, for example, if the measurement position was too high in the phase of the blood pressure and inclination measurement. Vice versa, a low forearm (e.g., a forearm directed downward in relation to the forearm at normal heart height) is displayed if the position of the wrist was too low in the phase of the blood-pressure measurement. Precise feedback may also be given about the measurement errors using arrows or other symbols or display means.

1. A method of determining blood pressure, the method comprising
applying an application unit to a body part, the application until including a pressure sensor that generates pressure signals;
generating one or more signals indicative of position, movement or acceleration of the body part while the application unit is generating the pressure signals; and
determining blood pressure with an analysis unit as a function of at least the pressure signals;
wherein the one or more signals indicative of position, movement and acceleration include at least four discrete signals that are each used for determining the blood pressure or evaluating blood-pressure measurement as a function of position, movement or acceleration of the body part during pressure sensing.

2. The method according to claim 1, further comprising:
detecting at least four pressure signals;
assigning the at least four discrete signals to the at least four pressure signals; and
each pressure signal is used to determine blood pressure as a function of a good/bad analysis of its assigned discrete signal.

3. (canceled)

4. The method according to claim 1, comprising
ignoring the pressure signals corresponding to which one or more of the signals indicative of position, movement, or acceleration of the body part fall either above or below particular limiting values when determining the blood pressure; and
determining the blood pressure on the basis of remaining detected pressure signals.

5. The method according to claim 1, further comprising displaying by the analysis unit whether the body part assumes a suitable orientation during the detection of the pressure signals as a function of a particular detected position, movement, or acceleration of the body part.

6. The method according to claim 1, further comprising controlling a measurement sequence for detecting the pressure signals as a function of a particular detected position, movement or acceleration of the body part.

7. The method according to claim 6, further comprising subjecting the body part to a pressure change cycle during the detection of the pressure signals and controlling the pressure change cycle as a function of the particular detected position, movement or acceleration of the body part.

8. The method according to claim 1, further comprising interrupting or delaying a provided pressure change if an associated body part position, movement, or acceleration signal lies either above or below particular limiting values, and is reassumed if the associated signal returns back into particular setpoint ranges.

9. The method according to claim 1, further comprising controlling the measurement sequence for detecting the pressure signals as a function of a time duration within which the one or more signals indicative of the position, movement, or acceleration of the body part lie outside particular setpoint ranges.

10. The method according to claim 1, further comprising displaying, either during the blood-pressure measurement or after the blood-pressure measurement or after a termination of the blood-pressure measurement whether the position, movement, and/or acceleration of the body part during the blood-pressure measurement resulted in valid or invalid blood-pressure measurement results.

11. The method according to claim 10, further comprising additionally displaying, in response to an invalid blood-pressure measurement, whether the body part was too high or too low in relation to heart height, or whether the body part was moved during the blood-pressure measurement.

12. The method according to claim 10, comprising representing the valid or invalid blood-pressure measurement on the basis of a human torso having a graphically emphasized body part.

13. A blood-pressure measuring device comprising:
a pressure sensor that generates pressure signals;
an application unit configured to apply the pressure sensor to a body part;
an analysis unit that determines the blood pressure as a function of at least the pressure signals of the pressure sensor, and
an orientation detection unit that detects signals indicative of the position, movement, or acceleration of the body part, wherein the analysis unit considers the detected signals indicative of position, movement, or acceleration of the body part during the blood pressure determination,
wherein the orientation detection unit detects at least four discrete signals indicative of the position, movement, or acceleration of the body part, and the analysis unit analyzes the blood pressure as a function of each discrete signal during pressure sensing.

14. The blood-pressure measuring device according to claim 13, wherein the analysis unit distinguishes between good and bad detected pressure signals as a function of an analysis of discrete signals for position, movement or acceleration associated with each pressure signal.

15. The blood-pressure measuring device according to claim 13, wherein the orientation detection unit detects each discrete signal indicative of the position, movement or acceleration of the body part using signals in temporal association with each pressure signal, and the analysis unit analyzes the pressure signals as a function of their associated, respective discrete signals.

16. The blood-pressure measuring device according to claim 13 wherein the analysis unit comprises
a memory that stores one or both of limiting values and setpoint ranges for the signals indicative of position, movement or acceleration of the body pair during the pressure signal detection; and
a comparison unit that compares the detected signals indicative of the position, movement, or acceleration of the body part to the particular limiting values,
wherein the analysis unit ignores the pressure signals for which the detected signals indicative of position, movement or acceleration of the body part lie either above or below the particular limiting values when determining the blood pressure, and determines the blood pressure as a function of remaining detected pressure signals.

17. The blood-pressure measuring device according to claim 13 further comprising a display unit activated by the analysis unit as a function of a detected position, movement, or acceleration of the body part.

18. The blood-pressure measuring device according to claim 13 further comprising a measurement sequence control unit that controls a measurement sequence for detecting the pressure signals as a function of a particular detected position, movement, and/or acceleration of the body part.

19. The blood-pressure measuring device according to claim 18, further comprising a pressure control unit activated by the measurement sequence control unit, the measurement sequence control unit controlling the pressure control unit as a function of the signals detected by the orientation detection unit.

20. The blood-pressure measuring device according to claim 18, wherein the measurement sequence control unit interrupts and/or delays a pressure change if an associated body part position, movement, or acceleration signal lies either above or below particular limiting values.

21. The blood-pressure measuring device according to claim 18, wherein the measurement sequence control unit comprises a time detection unit that detects a time duration within which any one or more of the position, movement, or acceleration of the body part detected by the orientation detection unit lie outside stored setpoint ranges, and wherein the measurement sequence control unit terminates a measurement cycle if the time duration detected by the time detection unit is greater than a predefined time duration.

22. The blood-pressure measuring device according to claim 16 further comprising a validation display that displays an output of the comparison unit, and a good/bad statement on the position, movement, or acceleration of the body part during the blood-pressure measurement.

23. The method of claim 9, wherein the measuring sequence is interrupted if the detected one or more signals indicative of the position movement or acceleration of the body part lie outside particular setpoint ranges longer than a predefined time duration.
